(19) United States
(12) Patent Application Publication
(10) Pub. No.: US 2017/0143855 A1
(43) Pub. Date: May 25, 2017

(54) GEL COMPOSITION FOR ULTRASOUND PROCEDURE AND RELATED METHODS

(71) Applicant: Access Business Group International LLC, Ada, MI (US)

(72) Inventor: Louise M. Schneider, Rockford, MI (US)

(21) Appl. No.: 15/354,164

(22) Filed: Nov. 17, 2016

Related U.S. Application Data
(60) Provisional application No. 62/258,090, filed on Nov. 20, 2015.

(51) Int. Cl. A61K 49/22

(52) U.S. Cl. CPC A61K 49/226

(57) ABSTRACT

A gel composition for an ultrasound procedure comprises: (a) a vehicle; (b) a chelating agent; (c) an anti-inflammatory agent; (d) a thickening agent; (e) a skin-conditioning agent; (f) a humectant; (g) a preservative; and (h) a neutralizer. A method of preparing the gel composition and a method of applying the gel composition for an ultrasound procedure are also disclosed.

### FIELD OF THE INVENTION

The present invention generally relates to a gel composition, and, more specifically, to a gel composition for an ultrasound procedure and to related methods.

### DESCRIPTION OF THE RELATED ART

Compositions are well known in the art and utilized in myriad industries and end use applications. One specific example of such an end use application is an ultrasound procedure (e.g. sonography). During ultrasonic imaging, a probe or transducer, which emits sound waves, is placed adjacent a subject's body, and the sound waves are utilized for various purposes. For example, ultrasonic imaging may be utilized for identifying or mapping internal aspects of the patient's body, such as muscles, tendons, organs, a fetus, etc. Further, ultrasound procedures are increasingly utilized for treating skin conditions unrelated to ultrasonic imaging. For example, an ultrasound procedure may be utilized to reduce wrinkles or otherwise improve skin conditions, e.g. on a subject's face and/or neck. A gel composition is typically utilized as the interface between the probe or transducer and the patient's skin.

Conventional gel compositions suffer from a number of drawbacks. For example, certain conventional gel compositions result in sound wave reflections due to poor acoustic properties, which deleteriously impact the resulting imaging. Other conventional gel compositions do not provide suitable lubricating properties, or prematurely dry when exposed to ambient air or have an undesirable aesthetic or feel. Further still, certain conventional gel compositions, while providing desirable lubricating properties, have low viscosities, which result in difficulty in maintaining a localized geography on the subject's body, particularly if the subject is in a vertical position for the ultrasound procedure.

### BRIEF SUMMARY OF THE INVENTION

The present invention provides a gel composition for an ultrasound procedure. The gel composition comprises: (a) a vehicle in an amount of from 70 to 95 weight percent (wt. %); (b) a chelating agent in an amount of from greater than 0 to 0.1 wt. %; (c) an anti-inflammatory agent in an amount of from greater than 0 to 0.3 wt. %; (d) a thickening agent in an amount of from greater than 0 to 1.0 wt. %; (e) a skin-conditioning agent in an amount of from greater than 0 to 25 wt.%; (f) a humectant in an amount of from greater than 0 to 25 wt. %; (g) a preservative in an amount of from greater than 0 to 4 wt. %; and (h) a neutralizer in an amount of from greater than 0 to 1 wt. %, each based on the total weight of the gel composition.

The present invention also provides a method of preparing the gel composition (the “preparation method”). The preparation method comprises combining at least the (a) vehicle, the (b) chelating agent, the (c) anti-inflammatory agent, the (d) thickening agent, the (e) skin-conditioning agent, the (f) humectant, the (g) preservative and the (h) neutralizer to prepare the gel composition.

Further, the present invention provides a method of applying the gel composition for an ultrasound procedure (the “application method”). The application method comprises administering to skin of a subject the gel composition.

### DETAILED DESCRIPTION

A gel composition for an ultrasound procedure is disclosed, along with a method of its preparation and a method of its application to skin of a subject. The gel composition has excellent physical properties, including acoustic properties, and thus is well suited for ultrasound procedures. For example, the gel composition may be utilized on the skin of the subject at the interface between an ultrasonic probe or transducer and the skin. However, the gel composition is suitable for other end use applications separate from ultrasound procedures and is not limited in its end use or application.

The gel composition comprises (a) a vehicle, which is generally majority component of the gel composition. The (a) vehicle is a carrier for the components of the gel composition, and may be any vehicle, which serves to carry, alternatively partially solubilize, alternatively solubilize, or otherwise mix with the components of the gel composition. In specific embodiments, the vehicle comprises water. The gel composition may comprise a vehicle other than water in addition to water, i.e., a combination of different vehicles. In other embodiments, the (a) vehicle is water.

When the (a) vehicle comprises water, the water of the (a) vehicle of the gel composition may be from any source and may optionally be purified. For example, the water may be from a natural source, a municipal source, etc. Examples of purification methods include filtering/filtration, distillation, deionization, demineralization, a reverse-osmosis process, etc. Typically, the water of the gel composition is purified to remove any trace contaminants therefrom.

The (a) vehicle is present in the gel composition in an amount of from 70 to 95, alternatively from 72 to 93, alternatively from 74 to 91, alternatively from 76 to 89, alternatively from 78 to 87, alternatively from 80 to 85, weight percent (wt.%) based on the total weight of the gel composition. The amount of the (a) vehicle in the gel composition may vary based on the selection and relative amounts of other components, including optional components, in the gel composition.

The gel composition further comprises (b) a chelating agent. Combinations of different chelating agents may be utilized together as the (b) chelating agent. The (b) chelating agent of the gel composition is not limited.

The (b) chelating agent generally sequesters any metal ions that may be present in the gel composition, e.g. if inherently present in nominal amounts in the (a) vehicle, or if otherwise introduced via other components or from being present in ambient conditions upon formation of the gel composition. Specific examples of suitable chelating agents include alkali and/or alkaline earth metal salts, e.g. those of ethylene diamine tetraacetic acid and/or nitrilotriacetate. Specific species thereof include sodium EDTA; disodium EDTA; tetrasodium EDTA; dilithium EDTA; cesium EDTA; disodium magnesium EDTA; disodium calcium EDTA; monosodium nitrilotriacetate; disodium nitrolotriacetate; etc. Combinations of different chelating agents may be utilized together. In one embodiment, the (b) chelating agent comprises disodium EDTA.

The (b) chelating agent is present in the gel composition in an amount of from greater than 0 to 0.1, alternatively from 0.1 to 0.9, alternatively from 0.2 to 0.8, alternatively from 0.3 to 0.7, alternatively from 0.4 to 0.6, wt.% based on the total weight of the gel composition. The amount of the (b) chelating agent in the gel composition may vary based on the selection and relative amounts of other components, including optional components, in the gel composition.

In addition, the gel composition comprises (c) an anti-inflammatory agent. Combinations of different anti-inflammatory agents may be utilized together as the (c) anti-inflammatory agent. Examples of anti-inflammatory agents suitable for the gel composition include aloe vera, methylsulfonylmethane (MSM), emu oil, chondroitin, glucosamine, a capsaicinoid, arnica extract, grape seed extract, coriander oil, marigold extract, nettle leaf extract, Roman chamomile oil, blue-bottle extract, St. John's wort, willow bark extract, witch hazel extract, feverfew extract, barley grass, black cohosh, black snakeroot, bugbane, squawroot, Boswellia, borage, bromelain, burdock, calendula, cayenne, dandelion, devil's claw root, DHEA (dehydroepiandosterone), Echinacea, elderflower, evening primrose oil, flaxseed, ginkgo, ginger, ginseng, Hawthorne, kaempferol, licorice (e.g. licorice extract), life root, golden Senecio, squaw weed, golden groundsel, cocash weed, coughweed, ragwort, golden ragwort, grundy swallow, linden, marjoram, meadow sweet, NDGA, neem, Padma 28, quercetin, shea butter, turmeric, wild yam, wormwood, yucca, bisabolol, sucralfate, LIPACIDE, gauaiazulene, essential fatty acids, poly-unsaturated fatty acid derivatives from plant seed oils and other vegetable sources, or any other anti-inflammatory agent. Essential fatty acids (EFAs) may include omega-3 and omega-6 fatty acids such as linolenic acid and alpha linolenic acid. In addition, any known herbs or various agents that contain EFAs may be included in the gel composition as or with the (c) anti-inflammatory agent. Examples of such herbs include flaxseed and evening primrose oil.

In specific embodiments, the (c) anti-inflammatory agent of the gel composition comprises a licorice extract. As used herein, “licorice extract” refers to any compound or combination of compounds in the glycyrrhiza family (i.e., Glycyrrhiza glabra), including glycyrrhiza, glycerrhetic acid (also known as “enoxolone,” “uralenic acid,” and “glycyrrhetinic acid”), glycyrrhizic acid (also known as “glycyrrhizin,” “glycyrrhizinic acid,” and “glycyrrhetinic acid glycoside”), derivatives thereof, and combinations thereof. Specific examples of derivatives of licorice extracts include salts (e.g., metal salts, ammonium salts, and the like) and esters (e.g. saturated fatty acid esters, unsaturated fatty acid esters, diacid half esters, glycoside esters, and the like).

In certain embodiments, the (c) anti-inflammatory agent comprises a licorice extract ester derivative, such as saturated and/or unsaturated esters of glycerrhetic acid and glycyrrhizic acid. Representative licorice extract ester derivatives for use in the gel composition include monoammonium glycyrrhizinate, monopotassium glycyrrhizinate, dipotassium glycyrrhizinate, 1-beta-glycyrrhetic acid, stearyl glycyrrhetinate, 3-stearyloxy-glycyrrhetinic acid, disodium 3-succinyloxy-beta-glycyrrhetinate, and combinations thereof. In one embodiment, the (c) anti-inflammatory agent comprises dipotassium glycyrrhizinate.

The (c) anti-inflammatory agent is present in the gel composition in amount of from greater than 0 to 0.3, alternatively from 0.01 to 0.3, weight percent based on the total weight of the gel composition. As with the other components, the relative amount of the (c) anti-inflammatory agent in the gel composition may vary based on a number of factors, including its selection and the presence/absence of various optional components.

The gel composition further comprises (d) a thickening agent. Combinations of different thickening agents may be utilized together as the (d) thickening agent. The (d) thickening agent imparts the gel composition with a desired viscosity or otherwise modifies rheological properties such that the gel composition is a gel. In certain embodiments, the (d) thickening agent may alternatively be referred to as a thixotroping agent.

The (d) thickening agent is not limited and generally is utilized to increase a viscosity or otherwise modify rheological properties of the gel composition. The (d) thickening agent may be polymeric and/or water-absorbing material. One example of a thickening agent suitable for the gel composition is an acrylic acid polymer. For example, the (d) thickening agent may comprise a polymer of acrylic acid, which may be crosslinked, e.g. with an allyl ether. Specific examples of commercially available thickening agents include those sold under the Carbomer™ tradename, commercially available from Ashland Inc. of Covington, Ky. In one embodiment, the (d) thickening agent comprises Carbomer™ 980, commercially available from Ashland Inc. of Covington, Ky.

Additional examp.les of thickening agents include acrylamide copolymers, acrylate copolymers and salts thereof (such as sodium polyacrylate), xanthan gum and derivatives, cellulose gum and cellulose derivatives (such as methylcellulose, methylhydroxypropylcellulose, hydroxyethylcellulose, hydroxypropylcellulose, polypropylhydroxyethylcellulose), starch and starch derivatives (such as hydroxyethylamylose and starch amylase), polyoxyethylene, carbomer, alginates (such as sodium alginate), arabic gum, cassia gum, carob gum, scleroglucan gum, gellan gum, rhamsan gum, karaya gum, carrageenan gum, guar gum and guar gum derivatives, cocamide derivatives (including cocamidopropyl betaine and cocamide MIPA), and mixtures thereof.

In various embodiments, the (d) thickening agent is present in the gel composition in an amount of from greater than 0 to 1, alternatively from 0.05 to 1, alternatively from 0.2 to 0.9, alternatively from 0.4 to 0.8, alternatively from 0.6 to 0.7, weight percent based on the total weight of the gel composition. As with the other components, the relative amount of the (d) thickening agent in the gel composition may vary based on a number of factors. When the gel composition further comprises a (h) neutralizer for neutralizing the (d) thickening agent, as described below, these amounts are on a pre-reaction or pre-neutralization basis of the gel composition.

The gel composition additionally comprises (e) a skin-conditioning agent. Combinations of different skin-conditioning agents may be utilized together as the (e) skin-conditioning agent. The (e) skin-conditioning agent may serve a variety of functions in the gel composition, including imparting the gel composition with soothing properties when applied to the skin of the subject. Further, depending on a selection of the (e) skin-conditioning agent, the (e) skin-conditioning agent may also have properties similar to humectants and emollients.

The (e) skin-conditioning agent may impart or restore moisture to skin. Increasing skin water content may make the skin softer and more pliable. Skin-conditioning agents may serve to mimic the action of normal skin secretions in maintaining suppleness in the skin and provide a barrier to evaporation. Conventional skin-conditioning agents generally include two general types: occlusives and humectants. Occlusive moisturizers form a layer on the skin which reduces the rate of evaporation. Humectants are nonocclusive hygroscopic substances which retain water and make the water available to the skin. Humectants may also function by improving the lubricity of the skin.

The (e) skin-conditioning agent may comprise a single moisturizing ingredient or it may comprise a plurality of ingredients which may be included to serve diverse purposes such as emollients, emulsifiers, lipids, surfactants, thickeners, and preservatives. Further, the (e) skin-conditioning agent may have both occlusive and nonocclusive properties. Water may be present along with the ingredients included in a skin-conditioning agent. Selection of the levels and types of skin-conditioning agents incorporated in the gel composition may be made without adversely affecting its stability or in-use characteristics.

Suitable skin-conditioning agents include long chain C₁₂-C₂₂ fatty acids, liquid water-soluble polyols, glycerin, propylene glycol, sorbitol, polyethylene glycol, ethoxylated/propoxylated ethers of methyl glucose, ethoxylated/propoxylated ethers of lanolin alcohol, lanolin alcohol, coconut fatty acid, tallow fatty acid, nonocclusive liquid water-soluble polyols, aloe vera gel, aloe vera gel condensed, aloe vera gel freeze-dried powder, aloe vera gel oil extract, amino acids, amniotic fluid, avocadin, calcium protein complex, cashew oil, chia oil, chitin, chitosan, chitosan PCA, cholesteric esters, chondroitin sulfate, collagen, collagen amino acids, copper protein complex, dioctyl maleate, dipentaerythritol fatty acid ester, elastin, ethyl panthenol, evening primrose oil, glycereth-12, glycosphingo lipids, hyaluronic acid, hybrid safflower oil, hydrogenated polyisobutene, hydrolyzed collagen, hydrolyzed elastin, hydrolyzed fibronectin, hydrolyzed mucopolysaccharides, hydrolyzed silk, hydrolyzed wheat protein, jojoba esters, keratin amino acids, kiwi fruit extract, lactamide MEA, liposomes, live yeast cell derivative liposome, marina polyaminosaccharide, mineral oil, mink oil ethyl ether, mucopolysaccharides, mucopolysaccharides, palmetto extract, leontopodium alpinum extract (Edelweiss), pantethine, paraffin, PEG-4, PEG-6, PEG-8, PEG-12, PEG-100 stearate, perfluoropolymethyl-isopropyl ether, petrolatum, petroleum wax, pistachio oil, placenta extract, plankton extract, polyamino sugar condensate, polybutene, polyglyceryl methacrylate, polypentaerythrityl tetralaurate, PPG-10 butanediol, PPG-20 methyl glucose ether distearate, royal jelly extract, saccharide isomerate, selenium protein complex, serum albumin, sodium hyaluronate dimethylsilanol, sodium lactate methylsilonol, sodium mannuronate methylsilanol, soluble collagen, super oxide dismutase, super oxide dismutase liposome, tissue extract, tocopheryl linoleate, lipophylic moisturizers such as lysolecithin, lecithin, cholesterol, sitosterol, cholesterol esters, sphingolipids, or ceramides, low molecular moisturizer such as serine, glutamine, sorbitol, mannitol, glycerin, sodium pyrrolidone-carboxylate, butylene glycol, propylene glycol, lactic acid, or lactic acid salts, high molecular moisturizers such as hyaluronic acid, sodium hyaluronate, elastin, alginic acid, mucopolysaccharides, polyethylene glycol, polyaspartic acid salts, or water soluble chitin, hydrocarbon oils, hydrocarbon waxes, silicones (e.g. dimethicone), bis-PEG-18 methyl ether dimethyl silane wax, fatty acid derivatives, cholesterol, cholesterol derivatives, di-and tri-glycerides, vegetable oils, vegetable oil derivatives, liquid nondigestible oils, combinations of liquid digestible or nondigestible oils with solid polyol polyesters, acetoglyceride esters, alkyl esters, alkenyl esters, lanolin and its derivatives, milk tri-glycerides, wax esters, glyceryl esters and derivatives (e.g. PEG-7 glyceryl cocoate), beeswax derivatives, sterols, phospholipids, or any other skin-conditioning agent.

Additional examples of suitable skin-conditioning agents, e.g. occlusive skin-conditioning agents, include petrolatum, paraffin, waxes, greases, mineral oil, beeswax, lanolin and oil-soluble lanolin derivatives, saturated and unsaturated fatty alcohols such as behenyl alcohol, squalene, various animal and vegetable oils such as almond oil, apricot oil, apricot pit oil, avocado oil, cade oil, castor oil, cinnamon oil, corn oil, cottonseed oil, evening primrose oil, grape oil, grape seed oil, hazelnut oil, jojoba oil, linseed oil, liver oil, macadamia nut oil, mink oil, neetsfoot oil, olive oil, palm kernel oil, palm nut oil, palm oil, peach pit oil, peanut oil, pine oil, pistachio nut oil, poppy seed oil, rapeseed oil, rice bran oil, rice germ oil, safflower oil, sasanqua oil, sesame oil, sesame seed oil, soybean oil, sunflower oil, sunflower seed oil, tsubaki oil, walnut oil, wheat germ oil, wheat germ oil, tea seed oil, triglycerine, glycerine trioctanate, glycerine triisopalmitate, cacao fat, beef tallow, sheep fat, hog fat, horse fat, hydrogenated oil, hydrogenated castor oil, Japanese wax, shea butter, beeswax, candelilla wax, cotton wax, carnauba wax, bayberry wax, tree wax, spermaceti, montan wax, bran wax, lanolin, reduced lanolin, hard lanolin, kapok wax, sugarcane wax, jojoba wax, shellac wax, or any other skin-conditioning agent exhibiting occlusive properties.

The (e) skin-conditioning agent may include agents that mimic natural ingredients and function as botanicals, including vitamins, hydroxy acids, and retinoids. Vitamins may include vitamin A, retinol, retinol palmitate, inositol, pyridoxine chlorate, benzyl nicotinate, nicotinamide, dlα-tocopheryl nicotine, magnesium ascorbyl phosphate, tetrahexyldecyl ascorbate, vitamin D₂ (ergocalciferol), dlα-tocopherol, potassium dl-α-tocopherol-2-L-ascorbic diester, dl-α-tocopheryl acetate, pantothenic acid, biotin, or any other vitamin. Some ingredients that may reduce the severity of dry skin are alpha hydroxy acids (AHA) and beta hydroxy acids (BHA), including their salts, as well as retinoids. The hydroxy acids are classified according to the number of carboxylic acids on their configuration. Monocarboxylic acids are glycolic, lactic, and mandelic acids. Dicarboxylic acids include malic and tartaric acids. Tricarboxylic acids embody citric acid found in citrus fruits. The BHAs encompass mostly salicylic acid and its derivatives. AHAs have been shown to exfoliate. Thus, they are useful in hyperkeratotic conditions. Other ingredients of a skin-conditioning agent may include elastin, lecithin, sodium hyaluronate, sodium passive cutaneous anaphylaxis, ceramides, naturally occurring skin lipids and sterols, artificial or natural oils, humectants, emollients, emulsifiers, preservatives, lubricants, greases, natural moisturizing factors (NMF) including low molecular weight substances such as ammonia, amino acids, glucosamine, creatinine, citrate and ionic solutions such as sodium, potassium, chloride, phosphate, calcium and magnesium, sodium pyrrolidone carboxylic acid, hexadecyl, myristyl, isodecyl, or isopropyl esters of adipic, lactic, oleic, stearic, isostearic, myristic and linoleic acids, and their corresponding alcohol esters, sodium isostearoyl-2-lactylate and sodium capryl lactylate, glycerin, polyethylene glycol, propylene glycol, sorbitol, polyethylene glycol and propylene glycol ethers of methyl glucose, polyethylene glycol and propylene glycol ethers of lanolin alcohol, lactic acid, L-proline, and other free fatty acids, coconut fatty acid, tallow fatty acid, nonocclusive liquid water-soluble polyols and the essential amino acid compounds found naturally in the skin, and stearic and lauric acids.

The (e) skin-conditioning agent may be present in amount of from greater than 0 to 25, alternatively from 5 to 20, weight percent based on the total weight of the gel composition. As with the other components, the relative amount of the (e) skin-conditioning agent in the gel composition may vary based on a number of factors.

In specific embodiments, the (e) skin-conditioning agent comprises a combination of skin-conditioning agents. In this specific embodiment, the (e) skin-conditioning agent comprises at least two, alternatively at least three, alternatively at least four, different skin-conditioning agents. For example, the (e) skin-conditioning agent may comprise a combination of butylene glycol, a cosmetic wax, a glyceryl ester, and a polysaccharide.

The cosmetic wax may be any suitable cosmetic wax. In certain embodiments, the cosmetic wax is at least partially soluble or dispersible, alternatively fully soluble or dispersible, in water. Alternatively, the cosmetic wax may be utilized in combination with a carrier vehicle other than water, which may at least partially solubilize or disperse the cosmetic wax. The cosmetic wax may be organic and may additionally include inorganic portions, e.g. a silane portion. One specific example of an exemplary cosmetic wax suitable for the gel composition is a bis-PEG-18 methyl ether dimethyl silane wax.

The glyceryl ester may be, for example, PEG-7 glyceryl cocoate.

The polysaccharide may be derived from any source, e.g. by fermentation of carbohydrates, such as sorbitol, glucose, etc. One specific example of a suitable polysaccharide is biosaccharide gum-1.

In these specific embodiments, the butylene glycol may be present in the gel composition in an amount of from 1 to 15, alternatively from 1 to 10, alternatively from 2 to 6, weight percent based on the total weight of the gel composition. The cosmetic wax may be present in the gel composition in an amount of from 1 to 10, alternatively from 1 to 7, alternatively from 2 to 4, weight percent based on the total weight of the gel composition. The glyceryl ester may be present in the gel composition in an amount of from greater than 0 to 10, alternatively from greater than 0 to 5, alternatively from 0.5 to 2.5, weight percent based on the total weight of the gel composition. The polysaccharide may be present in the gel composition in an amount of from 1 to 20, alternatively from 1 to 15, alternatively from 1 to 10, weight percent based on the total weight of the gel composition.

The gel composition further comprises (f) a humectant. Combinations of different humectants may be utilized together as the (f) humectant. The (f) humectant of the gel composition is not limited. The (f) humectant is distinguished and different from the (e) skin-conditioning agent.

Examples of suitable humectants include, but are not limited to, glycerin, hyaluronic acid, sorbitol, urea, alpha hydroxy acids, sugars, lactic acid, propylene glycol, glyceryl triacetate, lithium chloride, polyols like sorbitol, xylitol and maltitol, polymeric polyols like polydextrose, natural extracts like quillaia, hexadecyl, myristyl, isodecyl and isopropyl esters of adipic, lactic, oleic, stearic, isostearic, myristic and linoleic acids, as well as many of their corresponding alcohol esters (e.g. sodium isostearoyl-2-lactylate, sodium capryl lactylate), hydrolyzed protein and other collagen-derived proteins, aloe vera gel, acetamide monoethanolamide (MEA), compounds found to be naturally occurring in the stratum corneum of the skin such as sodium pyrrolidone carboxylic acid, lactic acid, urea, L-proline, guanidine and pyrrolidone, acetamide MEA, acetamido propyl trimonium chloride, calcium stearoyl lactylate, chitosan pyrrolidone carboxylic acid (PCA), diglycerol lactate, ethyl ester of hydrolyzed silk, fatty quaternary amine chloride complex, glycereth-7, glycereth-12, glycereth-26, glycereth-4.5 lactate, glycerin, diglycerin, polyglycerin, hydrolyzed fibronectin, lactamide MEA, lactamide N-(2-hydroxyetheryl), mannitol, methyl gluceth-10, methyl gluceth-20, methylsilanol PCA, panthenol, PCA, polyethylene glycol (PEG), PEG-4, PEG-8, polyamino sugar condensate, quaternium-22, sea salts, sodium capryllactylate, sodium hyaluronate, sodium isostearoyl lactylate, sodium lactate, sodiumlauroyl lactylate, sodium PCA, sodium polyglutamate, sodium stearoyl lactylate, soluble collagen, sorbitan laurate, sorbitan oleate, sorbitan sesquiisostearate, sorbitan stearate, sorbitol, sphingolipids, TEA-PCA, ethylene glycol, diethylene glycol, triethylene glycol, and other polyethylene glycols, propylene glycol, dipropylene glycol and other propylene glycols, 1,3-butylene glycol, 1,4-butylene glycol and other butylene glycols, glycerol, diglycerol and other polyglycerols, mannitol, xylitol, maltitol and other sugar alcohols, glycerol ethylene oxide (EO) and propylene oxide (PO) adducts, sugar alcohol EO and PO adducts, adducts of EO or PO and monosaccharides such as galactose and fructose, adducts of EO or PO and polysaccharides such as maltose and lactose, sodium pyrrolidonecarboxylate, polyoxyethylene methyl glycoside, polyethylene glycol, propylene glycol, dipropylene glycol, 1,3-butylene glycol, hexylene glycol, xylitol, maltitol, maltose, D-mannitol, gluten, glucose, fructose, lactose, sodium chondroitin sulfate, sodium hyalonate, sodium adenosine phosphate, gallates, pyrrolidone carbonates, glucosamine, cyclodextrin, alpha hydroxy acids, 2-methyl-1,3-propane diol, or combinations thereof.

The (f) humectant may be present in amount of from greater than 0 to 25, alternatively from 1 to 15, alternatively from 1 to 10, weight percent based on the total weight of the gel composition. As with the other components, the relative amount of the (f) humectant in the gel composition may vary based on a number of factors.

In specific embodiments, the (f) humectant of the gel composition comprises a first humectant and a second humectant. Even in these embodiments, the (f) humectant may comprise humectants in addition to the first humectant and the second humectant. In these specific embodiments, the first humectant comprises glycerin, and the second humectant comprises sodium hyaluronate. The first humectant is typically utilized in a greater amount than the second humectant. For example, the first humectant may be present in the gel composition in an amount of from 1 to 15 weight percent, and the second humectant may be present in the gel composition in an amount of from greater than 0 to 1, alternatively from 0.01 to 1, weight percent, each based on the total weight of the gel composition. When the second humectant comprises sodium hyaluronate, the second humectant may be combined with water prior to being incorporated into the gel composition, as described below.

The gel composition also comprises (g) a preservative. Combinations of different preservatives may be utilized together as the (g) preservative. The (g) preservative of the gel composition is not limited.

Examples of suitable preservatives include grape seed extract, methylparaben, propylparaben, diazolindinyl urea, sorbic acid, phenoxyethanol, ethylparaben, butylparaben, sodium butylparaben, caprylyl glycol, dehydroacetic acid, chlorphenesin, or any other preservative. Combinations of different preservatives may be utilized.

In a specific embodiment, the (g) preservative of the gel composition may be a preservative premix. The preservative premix typically comprises a combination or two or more preservatives in a carrier. One such example comprises a combination of chlorphenesin and methylparaben (each present in the preservative premix in an amount of 7.5 percent by weight based on the total weight of the preservative premix) in a carrier comprising a combination of glycerin and butylene glycol (each present in the preservative premix in an amount of 42.5 percent by weight based on the total weight of the preservative premix).

In specific examples, the (g) preservative is present in the gel composition in an amount of from greater than 0 to 4, alternatively from 1 to 4, weight percent based on the total weight of the gel composition. The amount of the (g) preservative in the gel composition may vary based on the selection and relative amounts of other components, including optional components, in the gel composition. When the preservative premix is utilized as the (g) preservative, the weight of the carrier is typically included in the relative amount of the (g) preservative in the gel composition. Further, when the preservative premix is utilized as the (g) preservative, the preservative premix may be formed in situ in the gel composition or may be provided and combined as the preservative premix. Typically, the gel composition is formed with the preservative premix itself being a discrete component, as opposed to being formed in situ.

The gel composition further comprises (h) a neutralizer. The (h) neutralizer is generally for neutralizing the (d) thickening agent, particularly when the (d) thickening agent is acidic, e.g. the acrylic acid polymer. Alternatively, the neutralizer may be a buffering agent or alternatively utilized for modifying pH of the gel composition.

The (h) neutralizer may be any compound suitable for neutralizing an acid. One specific example of a suitable (h) neutralizer is a trialcoholamine, e.g. triethanolamine. Other examples of suitable neutralizers include basic compounds, such as aliali metal hydroxides, e.g. potassium hydroxide (KOH), sodium hydroxide (NaOH), and ammonium hydroxide (NH₄OH). As described below in regards to the method, the (h) neutralizer may be disposed in or added to the other components when forming the gel composition after the (d) thickening agent so as to prevent premature neutralization.

In various embodiments, the (h) neutralizer is present in the gel composition in a catalytic amount, e.g. an amount of from greater than 0 to 1, alternatively from 0.05 to 1, alternatively from 0.2 to 0.9, alternatively from 0.3 to 0.9, weight percent based on the total weight of the gel composition. The amount of the (h) neutralizer in the gel composition may vary based on the selection and relative amounts of other components, including optional components, in the gel composition. If the (h) neutralizer reacts with the (d) thickening agent, as introduce above, these amounts are on a pre-reaction or pre-neutralization basis of the gel composition.

Optionally, the gel composition may include one or more additional components, such as additives. Suitable additives include those understood in the art, including but not limited to, oils, extracts, skin protectants, disinfectants, antiseptics, drugs and drug substances, analgesic compounds, anti-oxidants, sunscreens, insect repellants, exfoliants, fragrances, colors, fillers, solvents, carriers, other types of additives known to those of skill in the art, and combinations thereof. Such additives may be utilized alone or in combination.

The gel composition typically has a viscosity contingent on the relative amount of components in the gel composition and their selection. In certain embodiments, the gel composition has a viscosity of at least 10,000, alternatively at least 20,000, alternatively at least 30,000, alternatively at least 40,000, centipoise at 25° C. For example, the gel composition may have a viscosity from 20,000 to 150,000, alternatively from 30,000 to 120,000, alternatively from 40,000 to 80,000, centipoise at 25° C. The gel composition typically has a density greater than water, e.g. a specific gravity greater than 1. The gel composition typically has a pH in the range of 5 to 7, alternatively 5.7 to 6.6. pH may be modified by inclusion of optional components and modifying relative amount of, for example, the (h) neutralizing agent.

Modifying the relative amounts of the components in the gel composition impacts resulting physical properties of the gel composition, and thus the gel composition can be advantageously modified or optimized.

The gel composition has excellent physical properties and is well suited for various end uses and applications, particularly an ultrasound procedure. The gel composition is non-runny and can be applied to the skin of a subject when the subject is standing vertically or otherwise on non-horizontal substrates without running or dripping. As such, the gel composition is particularly well suited for ultrasound procedures, whether the subject is in a horizontal (laying) or vertical (standing) position, as described below. The gel composition also has excellent acoustic properties which do not interfere with soundwaves during such ultrasound procedures. Moreover, the gel composition has excellent lubricating properties, and thus may serve as a desirable interface between the skin of the subject and an instrument, e.g. an ultrasound probe or transducer. However, the gel composition is not so limited and may be utilized in end use applications other than ultrasound procedures. For example, the gel composition may be rubbed onto and/or into the skin separate from any ultrasound procedure.

The present invention also provides a method of preparing the gel composition (the “preparation method”). The preparation method comprises combining the (a) vehicle, the (b) chelating agent, the (c) anti-inflammatory agent, the (d) thickening agent, the (e) skin-conditioning agent, the (f) humectant, the (g) preservative and the (h) neutralizer to prepare the gel composition.

The components may be combined in the preparation method in any order, optionally under shear or mixing. Individual components may optionally be combined with one another to form various “premixes” of components prior to or at the time of forming the gelcomposition. The vehicle may be added incrementally when preparing the gel composition. The conditions associated with the method of preparation may be modified as desired, e.g. in regards to temperature, relative humidity, etc. As understood in the art, parameters associated with the method, including temperature and time, may vary upon scaling up or down various methods based on manufacturing volumes and other factors.

In specific embodiments of the preparation method, the some of the (a) vehicle is first disposed in a vessel along with the (b) chelating agent and the (c) anti-inflammatory agent under mixing to give a mixture, and the (d) thickening agent is disposed incrementally in the mixture to give a thickened mixture. Generally, the (d) thickening agent is disposed incrementally in the mixture at a rate to avoid agglomeration or clumping. The amount of the (a) vehicle utilized in the mixture may vary, but is typically less than half of the total amount of the (a) vehicle utilized in the gel composition. The (d) thickening agent may absorb some of the (a) vehicle present in the mixture as the (d) thickening agent is incrementally disposed in the mixture, and the mixture is sheared or otherwise mixed as the (d) thickening agent is incrementally disposed in the mixture until hydrated with the (a) vehicle. Both the mixture and the thickened mixture are typically mixed such that the mixture and the thickened mixture are homogenous, although the mixture and the thickened mixture may alternatively and independently be heterogeneous.

In these specific embodiments of the preparation method, an additional amount of the (a) vehicle and the (e) skin-conditioning agent are disposed in the thickened mixture to give a first composition. As introduced above, the (e) skin-conditioning agent may comprise a plurality of skin-conditioning agents in concert, and such a plurality of skin-condition agents are typically disposed together (or in series) to the thickened mixture when forming the first composition. For example, each skin-conditioning agent may be discretely disposed in the thickening mixture or each of the skin-conditioning agents may be combined with the additional amount of the (a) vehicle such that the additional amount of the (a) vehicle and the (e) skin-conditioning agent together are disposed in the thickened mixture.

Further, in these specific embodiments of the preparation method, the first composition is heated to an elevated temperature for a period of time. The elevated temperature is typically less than a boiling point temperature of the (a) vehicle and the first composition. Typically, the elevated temperature is from 50 to 100, alternatively from 60 to 90, alternatively from 75 to 80, ° C. The elevated temperature is often utilized when the (e) skin-conditioning agent comprises, for example, the cosmetic wax, which may have an elevated melting point temperature. The period of time is typically from greater than 0 to 60, alternatively from 5 to 20, alternatively from 10 to 15, minutes. After heating the first composition to the elevated temperature for the period of time, the first composition is generally cooled to a temperature less than the elevated temperature, e.g. from 30 to 60, alternatively from 40 to 45, ° C. The first composition may be actively cooled to this temperature, or heating may merely cease such that the first composition reaches this temperature upon natural cooling from ambient conditions.

In addition, in these specific embodiments of the preparation method, the (g) preservative and the (h) neutralizer are disposed in the first composition to give the gel composition. The first composition is typically thickened when disposing the (h) neutralizer therein. Typically, the first composition is mixed during the addition of the (h) neutralizer without incorporation of any excess air. The mixing may be done in inert conditions or under vacuum.

In certain embodiments when the (f) humectant comprises sodium hyaluronate, either alone or together with other humectants, the sodium hyaluronate is combined with some of the (a) vehicle prior to combining with other components to form the gel composition.

Further, the present invention provides a method of applying the gel composition for an ultrasound procedure (the “application method”). The application method comprises administering to skin of a subject the gel composition. The gel composition may be rubbed or wiped onto the skin via any manner, and may be removed by wiping from the skin, or may be rubbed into/onto the skin after the ultrasound procedure.

Administering the gel composition to the skin may be carried out via any technique. For example, the gel composition may simply be applied to the skin by the subject, or by another. The gel composition may be dispensed, spread, and/or applied on the skin, optionally while applying a force to spread or apply the gel composition. In certain embodiments, the substrate may also take the form of an article to carry and deliver the gel composition to the skin of the subject. These steps can be repeated as many times as desired to achieve the desired benefit.

The gel composition has excellent ultrasonic and lubricating properties and is particularly suited for ultrasound procedures. In such procedures, the gel composition is applied on the skin of the subject at an interface between the skin and an ultrasound probe or transducer. Sound waves transfer through the gel composition and may be utilized, for example, for imaging or mapping, or for treating skin conditions. In specific embodiments, the gel composition may be utilized for ultrasound procedures to reduce wrinkles or otherwise improve skin conditions, e.g. on a subject's face and/or neck.

In view of the excellent physical properties of the gel composition, the gel composition may be applied while the subject is standing or seated without undesirably running or separating. In contrast, most conventional ultrasound gels are applied to subjects while laying horizontally to prevent gravity from causing the conventional ultrasound gels from undesirably running. In addition, the gel compositions have benefits to skin based on

The following examples are intended to illustrate the current embodiments and are not to be viewed in any way as limiting to the scope of the same.

### EXAMPLES

### Example 1

### Gel Composition

A gel composition is prepared in accordance with the current embodiments. Table 1 below sets forth the components utilized to prepare the gel composition and their relative amounts.

TABLE 1

| Component | Amount (g) |
|---|---|
| Vehicle (part one) | 369.30000 |
| Chelating Agent | 0.5000 |
| Anti-Inflammatory Agent | 0.5000 |
| Thickening Agent | 6.5000 |
| Vehicle (part two) | 400.000 |
| Skin-Conditioning Agent 1 | 40.0000 |
| Humectant 1 | 30.0000 |
| Skin-Conditioning Agent 2 | 30.000 |
| Skin-Conditioning Agent 3 | 10.0000 |
| Preservative | 36.7000 |
| Neutralizer | 6.5000 |
| Skin-Conditioning Agent 4 | 20.000 |
| Vehicle (part three) | 49.75 |
| Humectant 2 | 0.25 |

Vehicle (parts one, two and three) is water.

Chelating agent comprises disodium EDTA.

Anti-Inflammatory Agent comprises dipotassium glycyrrhizate.

Thickening Agent comprises an acrylic acid polymer.

Skin-Conditioning Agent 1 comprises butylene glycol.

Humectant 1 comprises glycerin.

Skin-Conditioning Agent 2 comprises a bis-PEG-18 methyl ether dimethyl silane wax.

Skin-Conditioning Agent 3 comprises polyethylene glycol (7) glyceryl cocoate.

Preservative comprises a preservative premix of chlorphenesin/methylparaben in a carrier comprising a mixture of glycerin and butylene glycol.

Neutralizer is triethanolamine

Skin-Conditioning Agent 4 comprises biosaccharide gum-1.

Humectant 2 comprises sodium hyaluronate.

In the method, the Vehicle, the Chelating Agent, and the Anti-Inflammatory Agent are combined in a vessel under mixing. The Thickening Agent is disposed in the vessel incrementally until hydrated. The Vehicle (part two), the Skin-Conditioning Agent 1, the Humectant 1, the Skin-Conditioning Agent 2, and the Skin-Conditioning Agent 3 are disposed in the vessel. The vessel is heated to an elevated temperature of from 75-80° C. for about 10-15 minutes while mixing its contents. Then, the contents of the vessel are cooled to about 40-45° C. At this temperature, the Preservative and the Neutralizer are mixed into the contents of the vessel without incorporating air. The Vehicle (part three) is combined with the Humectant 2 to form a premix, and the premix is disposed in the vessel under mixing along with Skin-Conditioning Agent 4 to give the gel composition.

The gel composition has a viscosity of 53,504 centipoise in ambient conditions (i.e., 25° C.), a specific gravity of 1.019, and a pH of 6.30.

The terms “comprising” or “comprise” are used herein in their broadest sense to mean and encompass the notions of “including,” “include,” “consist(ing) essentially of,” and “consist(ing) of. The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “—” in a range of values is “to” or “through”; a “>” is “above” or “greater-than”; a “≧” is “at least” or “greater-than or equal to”; a “<” is “below” or “less-than”; and a “≦” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the current embodiments independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language, which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The current embodiments have been described herein in an illustrative manner, and it is to be understood that the terminology that has been used is intended to be in the nature of words of description rather than of limitation. Many modifications and variations of the present invention are possible in light of the above teachings. The current embodiments may be practiced otherwise than as specifically described within the scope of the appended claims. The subject matter of all combinations of independent and dependent claims, both single and multiple dependent, is herein expressly contemplated.

What is claimed is:

1. A gel composition for an ultrasound procedure, comprising:
(a) a vehicle in an amount of from 70 to 95 weight percent (wt. %);
(b) a chelating agent in an amount of from greater than 0 to 0.1 wt. %;
(c) an anti-inflammatory agent in an amount of from greater than 0 to 0.3 wt. %;
(d) a thickening agent in an amount of from greater than 0 to 1.0 wt. %;
(e) a skin-conditioning agent in an amount of from greater than 0 to 25 wt. %;
(f) a humectant in an amount of from greater than 0 to 25 wt. %;
(g) a preservative in an amount of from greater than 0 to 4 wt. %; and
(h) a neutralizer in an amount of from greater than 0 to 1 wt. %, each based on the total weight of said gel composition.

2. The gel composition of claim 1, wherein said (a) vehicle is present in an amount of from 80 to 95 wt. % based on the total weight of said gel composition.

3. The gel composition of claim 1, wherein said (a) vehicle is water.

4. The gel composition of claim 1, wherein said (f) humectant comprises a first humectant and a second humectant different from said first humectant.

5. The gel composition of claim 4, wherein said first humectant comprises glycerin and said second humectant comprises sodium hyaluronate.

6. The gel composition of claim 1, wherein said (g) preservative comprises a combination of chlorophenesin and methylparaben in a carrier.

7. The gel composition of claim 1, wherein said (d) thickening agent comprises an acrylic acid polymer.

8. The gel composition of claim 1, wherein said (c) anti-inflammatory agent comprises dipotassium glycyrrhizate.

9. The gel composition of claim 1 having a viscosity at 25° C. of from 30,000 to 120,000 centipoise.

10. The gel composition of claim 1 having a pH of from 5.7 to 6.6.

11. A method of preparing the gel composition of claim 1, said method comprising:
combining the (a) vehicle, the (b) chelating agent, the (c) anti-inflammatory agent, the (d) thickening agent, the (e) skin-conditioning agent, the (f) humectant, the (g) preservative and the (h) neutralizer to prepare the gel composition.

12. The method of claim 11, wherein some of the (a) vehicle is first disposed in a vessel along with the (b) chelating agent and the (c) anti-inflammatory agent under mixing to give a mixture, and the (d) thickening agent is disposed incrementally in the mixture to give a thickened mixture.

13. The method of claim 12, wherein an additional amount of the (a) vehicle and the (e) skin-conditioning agent are disposed in the thickened mixture to give a first composition, and wherein the first composition is heated to an elevated temperature.

14. The method of claim 13, wherein the (g) preservative and the (h) neutralizer are disposed in the first composition to give the gel composition.

15. A method of applying a gel composition for an ultrasound procedure, said method comprising the step of administering to skin of a subject the gel composition of claim 1.

16. The method of claim 15, wherein the subject is standing or seated.
